(19) United States
(12) Patent Application Publication
(10) Pub. No.: US 2018/0320230 A1
(43) Pub. Date: Nov. 8, 2018

(54) HIGH THROUGHPUT IDENTIFICATION OF T-CELL RECOGNITION ANTIGENS AND EPITOPES

(71) Applicant: ARIZONA BOARD OF REGENTS ON BEHALF OF ARIZONA STATE UNIVERSITY, Scottsdale, AZ (US)

(72) Inventors: Joshua LaBaer, Chandler, AZ (US); Karen Anderson, Scottsdale, AZ (US); Ji Qiu, Chandler, AZ (US); Sri Krishna, Tempe, AZ (US)

(21) Appl. No.: 15/770,323

(22) PCT Filed: Oct. 27, 2016
(86) PCT No.: PCT/US16/59003

§ 371 Date: Apr. 23, 2018

Related U.S. Application Data
(60) Provisional application No. 62/246,975, filed on Oct. 27, 2015.

(51) Int. Cl. C12Q 1/6881; G01N 33/569; G01N 33/554

(52) U.S. Cl. CPC C12Q 1/6881; G01N 33/554; G01N 33/56972

(57) ABSTRACT

Provided herein are methods of classifying antigens and epitopes as being recognized by an individual's cellular immune response. More particularly, provided herein are methods for unbiased determination of which antigens are recognized by a population of T cells.

### CROSS-REFERENCE TO RELATED APPLICATIONS

This application claims the benefit of U.S. Provisional Application No. 62/246,975, filed Oct. 27, 2015, which is incorporated herein by reference as if set forth in its entirety.

### STATEMENT REGARDING FEDERALLY SPONSORED RESEARCH OR DEVELOPMENT

Not applicable.

### FIELD OF THE DISCLOSURE

This application relates to methods of classifying antigens as being recognized by an individual's cellular immune response. More particularly, this document provides methods for unbiased determination of which antigens are recognized by a population of T cells.

### BACKGROUND

There are two major branches to the human adaptive immune response: the cellular immune response and the humoral (antibody) immune response. Both branches play critical roles in protecting individuals from a variety of pathogens; however, the relative roles of each of these branches varies depending on the pathogen. Notably, both branches also contribute to the development of autoimmune disorders. Responses by each of the branches are triggered by the recognition of specific antigens. The adaptive immune system possesses memory, such that re-exposure to an antigen already recognized by the immune system will trigger a stronger response.

A variety of methods are available to identify the target antigens and even the specific epitopes recognized by the humoral immune response, such as protein microarrays and phage display techniques. For example, antibody arrays can be used to identify the antigens that bind to them tightly. However, current methods are poorly suited for identifying the antigens that trigger cellular immune responses. The T cell activation process requires two signals to occur at once. First, the T cell must encounter its cognate antigen after it has been properly processed, which includes proteolysis into the appropriate peptides, and displayed on the surface of an APC. Second, the APC must also display the matching HLA type for the T cell. Thus, it is not possible to identify which antigens activate a T cell by contacting T cells to a surface-bound array of antigens.

Accordingly, there remains a need in the art for high-throughput, scalable methods for analyzing a subject's cellular immune response and for identifying and classifying antigens and epitopes recognized by a subject's T cell population at the level of a single cell.

### SUMMARY OF THE INVENTION

In a first aspect, provided herein is a method of classifying antigens recognized by a T cell population. The method comprises contacting antigen presenting cells (APCs) modified to express at least one preselected target antigen to a plurality of T cells, wherein contacting occurs under conditions sufficient to allow binding between a T cell that specifically recognizes the at least one preselected target antigen expressed by the modified APCs; (b) separating target antigen-expressing APCs activated by the contacting; (c) amplifying nucleic acids isolated from the activated APC cells; and (d) detecting nucleotide sequences encoding preselected target antigens from the amplified nucleic acids, wherein a preselected target antigen is classified as being recognized by T cells of the plurality if a nucleotide sequence encoding the preselected target antigen is detected among the amplified nucleic acids. Binding between the modified APCs and the T cell can comprise formation of an immune complex between the modified APCs and a T cell receptor on the T cell that recognizes the at least one preselected target antigen. Target antigen-expressing APCs activated by the contacting can be separated from non-activated APCs and maturing APCs. Separating can comprise flow cytometry. The modified APCs can present the at least one preselected target antigen. The modified APCs can be obtained by recombinant techniques.

The antigen presenting cell can be selected from the group consisting of a dendritic cell, a macrophage, a monocyte, and a B cell. The at least one preselected target antigen can be derived from at least one of a tumor cell, a virus, a bacterium, a fungus, a yeast, and a parasite. The at least one preselected target antigen ca be a pathogen associated target antigen. The pathogen can be a virus, bacterium, fungus, yeast, or parasite. The virus can be selected from the group consisting of cytomegalovirus (CMV), adenovirus, Epstein Barr virus (EBV), respiratory syncytial virus (RSV), herpes simplex virus 6 (HSV6), parainfluenza 3, influenza B, BK virus, and JC virus. The at least one preselected target antigen can be a tumor associated antigen or an auto-immune antigen. The at least one preselected target antigen can comprise an epitope. The T cell population can be obtained from a human individual. The T cell population can be produced in vitro. Detecting nucleotide sequences can comprise DNA sequencing. In some cases, the method further comprises determining the relative abundance of antigen-specific T cells in the uncontacted T cell population. The antigen presenting cells (APCs) can be derived from a healthy donor.

These and other features, aspects, and advantages described herein will become better understood upon consideration of the following drawings, detailed description, and appended claims.

### BRIEF DESCRIPTION OF THE DRAWINGS

FIG. 1 is a schematic representing an exemplary embodiment of a method of classifying antigens recognized by a T cell population.

FIGS. 2A-2B are schematics representing target antigen presenting cell (APC) detection by capturing cytokines on the surface of APCs.

FIGS. 3A-3E present data from flow cytometric analysis of APC-capture assay for the identification of target antigens following T-cell activation. (A-C) APCs with cytokine captured were six-fold higher when pulsed with CEF (CMV+EBV+Flu) viral peptide pool (C), compared to APCs alone (A) or APCs pulsed with DMSO control (B). (D) Quantifying cytokine-captured APC with different effector (T-cell) to target (APC) ratios. Background cytokine captured does not increase as a function of T-cell number. Data is representative of 3 biological replicates from a donor; error bars denote standard error of mean. (E) Specificity of the APC-IFNg Capture. Cytokine capture on APC-cell surface when CD8+ T-cells target 100% of APCs pulsed with CEF-peptide pool (Top panel), and when only 50% of APCs were CEF-pulsed; rest were DMSO “cold” targets (Bottom panel).

FIG. 4 presents a schematic of exemplary workflow for target antigen identification by propidium iodide (PI) uptake for detection of T-cell mediated cell-death on target cells.

FIGS. 5A-5D present flow cytometric analysis of a PI-uptake assay to identify antigens following T-cell targeting. Cells from an artificial APC cell line (K562) expressing the donor-HLA (HLA-A0201) were pre-labeled with Hoescht live cell dye and mixed with ex vivo-expanded, peptide-pulsed donor peripheral blood mononuclear cells (PBMCs). 100 uM propidium iodide (PI) was added to the media, and the cells were incubated for 4 hours at 37° C. with intermittent mixing. (A) EBV-BMLF1 peptide pulsed PBMCs targeting negative control DMSO-pulsed K562.A2 targets. (B) EBV-BMLF1 peptide pulsed PBMCs targeting BMLF1 peptide-pulsed K562.A2 targets. (C) CEF viral peptide pool pulsed PBMCs targeting negative control DMSO-pulsed K562.A2 targets. (D) CEF viral peptide pool pulsed PBMCs targeting CEF-pulsed K562.A2 targets. Effector (PBMC) to target ratio was 5:1.

FIG. 6 presents data demonstrating identification of antigen transfected target cells by PI uptake as determined by flow cytometry. Artificial APC (aAPC) K562 cell lines expressing HLA A0201 (left) were incubated without PBMCs; (middle) were peptide pulsed and incubated with CEF-viral peptide stimulated PBMCs; or (right) were FluM1 transfected and incubated with CEF-viral peptide stimulated PBMCs. PI uptake assay was performed as described for FIG. 5. Effector (PBMC) to target ratio was 5:1.

### DETAILED DESCRIPTION OF THE INVENTION

All publications, patents, and patent applications mentioned in this specification are herein incorporated by reference to the same extent as if each individual publication, patent, or patent application was specifically and individually indicated to be incorporated by reference.

Standard methods are poorly suited for identifying the antigens that trigger T cell responses, because measuring those responses requires the binding of an activated T cell to an antigen presenting cell (APC), which must display both a correct peptide from the antigen relevant for the bound T cell and an HLA molecule that matches the T cell. There are no known methods for the unbiased determination of which antigens are recognized by a population of T cells. At least in part, the methods and compositions provided herein are based on the recognition and appreciation that a critical element of antigen-induced T cell activation is the information conveyed by the antigen.

### Methods

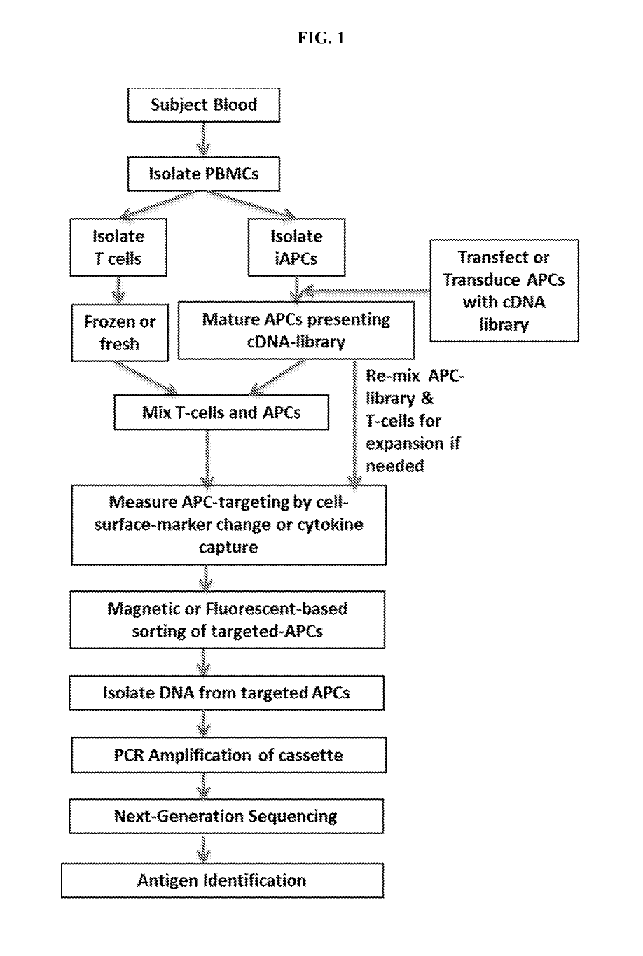

Accordingly, in a first aspect, provided herein is a method for determining identifying antigens recognized by a T cell population. An exemplary embodiment of the method is set forth in FIG. 1. Generally, methods provided herein comprise the following steps: (a) contacting antigen presenting cells (APCs) modified to express at least one preselected target antigen to a plurality of T cells, wherein contacting occurs under conditions sufficient to allow binding between a T cell that specifically recognizes the at least one preselected target antigen expressed by the modified APCs; (b) separating target antigen-expressing APCs activated by the contacting; (c) amplifying nucleic acids isolated from the activated APC cells; and (d) detecting nucleotide sequences encoding preselected target antigens from the amplified nucleic acids, wherein a preselected target antigen is classified as being recognized by T cells of the plurality if a nucleotide sequence encoding the preselected target antigen is detected among the amplified nucleic acids.

The method comprises obtaining antigen presenting cells that express candidate antigens. The term “antigen presenting cells” as used herein indicates immune cells whose main function is to process antigen material and present it on the surface to other cells of the immune system, thus functioning as antigen-presenting cells. Exemplary antigen presenting cells are dendritic cells, macrophages, B-cells and additional cells identifiable by a skilled person. As used herein, the term “antigen presenting cell” also includes vascular endothelial cells, microglia of the brain, and various epithelial and mesenchymal cell types.

Antigen presenting cells (APCs) for use according to the method provided herein can be of any origin, and in particular human origin. Appropriate sources of antigen presenting cells for use according to the methods described herein include an individual's own dendritic cells, macrophages, or other antigen presenting cells. In preferred embodiments, APCs are harvested, cultured in vitro, and induced to express candidate antigens using electroporation or lentiviral clones. Using an individual's APCs for the method enables clinical analysis of that individual's own T cell repertoire. While many tissue sources may be used, typical tissue sources comprise spleen, thymus, tissue biopsy, tumor, afferent lymph, lymph nodes, skin, GALT, bone marrow, apheresis or leukapheresis product, and/or peripheral blood. In some cases, apheresis product, bone marrow, and peripheral blood are preferred sources. Fetal tissue, fetal or umbilical cord blood, which is also rich in growth factors may also be used as a source of blood for obtaining precursor APC. Exemplary precursor cells may be, but are not limited to, embryonic stem cells, CD34+ cells, monocyte progenitors, monocytes, and pre-B cells.

Other appropriate sources for APCs include an established cell line having APC properties and having MHC/HLA type that matches the donor T cells (human or model animals), an established APC cell line that displays a highly common HLA type, an established APC cell line that is programmed to display an HLA type that matches the T cells, and an established APC cell line that has been engineered to produce a marker protein (e.g., eGFP, mCherry, luciferase, etc) upon induction by an activated T cell.

As used herein, the term “antigen” refers to any molecule (1) capable of being specifically recognized, either in its entirety or fragments thereof, and bound by the antigen-binding region of a antibody or its derivative; (2) containing peptide sequences which can be bound by MHC molecules and then, in the context of MEW presentation, can specifically engage its cognate T cell antigen receptor. By way of example, candidate antigens for expression in APCs as described herein represent processed antigens of a whole proteome, a collection of pathogen proteomes, or fragments of target antigens. In some cases, candidate antigens well suited for the methods provided herein are those polypeptide whose expressed is detectably altered (e.g., increased or reduced) as a result of a specific recognition of an epitope presented on the surface of an APC with certain MEW molecules.

In certain embodiments, APCs are modified to express one or more nucleic acids encoding polypeptides with unknown, potential target epitopes of reactive T-cells, like for example polynucleotides from a cDNA library or a genomic library. In some cases, the cDNA library is a species-specific, a pathogen-specific, a tissue-specific, a development-specific, or a subtractive library. The cloned polynucleotides can exert any length, e.g. they can comprise less than 20 nucleotides or, occurring more frequently, 20 to 100 nucleotides, but also 100 to 500, 500 to 1,500 nucleotides, but also up to 5,000 nucleotides, more rarely up to 10,000 nucleotides, but also more than 10,000 nucleotides.

In some cases, it will be advantageous to introduce a reporter gene construct into the APCs when introducing nucleotide sequences to induce expression of candidate antigens. For example, modified APCs comprise a reporter gene construct that provides a detectable signal if an APC is induced to mature or undergo apoptosis upon exposure to an activated T cell. Exemplary reporter constructs include, without limitation, constructs encoding eGFP, mCherry, or luciferase.

Any appropriate method of expressing candidate antigens can be used to modify APCs. Delivery of nucleotides sequences and/or expression constructs to target cells can be achieved in a variety of ways including transfection, transduction, electroporation, viral infection, encapsulation of the polynucleotide(s) in liposomes, and direct microinjection of the DNA into nuclei. As used herein, the term “transfection” refers to transient or stable introduction of exogenous molecules, such as nucleic acids, into cultured cells by various methods comprising chemical, biological or physical methods. “Transduction” refers to transient or stable introduction of exogenous material into eukaryotic cells using biological particles, such as viruses, as a carrier. In some embodiments, a transfection agent or delivery vehicle is used. As used herein, the terms “delivery vehicle,” refers to a compound or compounds that enhance the entry of oligonucleotides and polynucleotides into cells. Examples of delivery vehicles polynucleotides and/or expression constructs include, without limitation, protein and polymer complexes (polyplexes), combinations of polymers and lipids (lipopolyplexes), multilayered and recharged particles, lipids and liposomes (lipoplexes, for example, cationic liposomes and lipids), polyamines, calcium phosphate precipitates, polycations, histone proteins, polyethylenimine, polylysine, and polyampholyte complexes. One example of transfection reagent suitable for delivery of miRNA is siPORT™ NeoFX™ Transfection Agent (Ambion, Inc.), which can be used to transfect a variety of cell types. Nucleotide sequences can be readily electroporated into primary cells without inducing significant cell death. Nucleotide sequences can be transfected at various concentrations.

Transfection agents may be used to condense nucleic acids and/or to associate functional groups with a polynucleotide. Non-limiting examples of functional groups include cell targeting moieties, cell receptor ligands, nuclear localization signals, compounds that enhance release of contents from endosomes or other intracellular vesicles (such as membrane active compounds), and other compounds that alter the behavior or interactions of the compound or complex to which they are attached (interaction modifiers).

In some cases, candidate antigen expression is obtained using cDNA sequences cloned into vectors have one or more appropriate promoters and/or other cis-acting sequences that support protein expression in APCs. This might include APC-specific promoters or general transcription promoters. In some cases, candidate antigen constructs also comprise sequences that flank the cDNA that could be used later to amplify the DNA sequence using polymerase chain reaction (PCR). Optionally, a candidate antigen construct will comprise a selectable marker or fluorescent marker (e.g., GFP) for identification of APCs that comprise the construct.

Following introduction of sequences encoding candidate antigens, modified APCs are cultured under conditions adequate for expression, processing, and cell surface presentation of peptide antigens on the surface of the modified APCs.

The method further comprises exposing modified APCs, into which a library of genes that encode candidate antigens is introduced, to T cells. The term “exposing” as used herein refers to bringing into the state or condition of immediate proximity or direct contact. In some cases, therefore, modified APCs of the invention are contacted to T cells, where the T cells are able to recognize and bind to any modified APCs displaying relevant peptides. When this interaction occurs, the T cells will be activated, and they will in turn induce the APCs to further mature.

T cells can be obtained from a number of sources, including PBMC, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may also be obtained from a xenogeneic source, for example, from mouse, rat, non-human primate, and pig. In some cases, any number of T cell lines available in the art may be used.

T cell populations appropriate for use according to the methods described herein include, without limitation, experimentally produced T cells and T cells obtained from individuals having an infectious disease, from individuals having a known autoimmune disorder, from individuals having an identified cancer, or from healthy individuals. The term “infectious disease” as used herein, refers to any disease that is caused by an infectious organism or pathogen. Infectious organisms and pathogens may comprise viruses, (e.g., single stranded RNA viruses, single stranded DNA viruses, HIV, hepatitis A, B, and C virus, HSV, CMV EBV, HPV), parasites (e.g., protozoan and metazoan pathogens such as Plasmodia species, Leishmania species, Schistosoma species, Trypanosoma species), bacteria (e.g., Mycobacteria, in particular, M. tuberculosis, Salmonella, Streptococci, E. coli, Staphylococci), fungi (e.g., Candida species, Aspergillus species), Pneumocystis carinii, and prions (known prions infect animals to cause scrapie, a transmissible, degenerative disease of the nervous system of sheep and goats, as well as bovine spongiform encephalopathy (BSE) and feline spongiform encephalopathy of cats. Four prion diseases known to affect humans are (1) kuru, (2) Creutzfeldt-Jakob Disease (CJD), (3) Gerstmann-Straussler-Scheinker Disease (GSS), and (4) fatal familial insomnia (FFI)). As used herein “prion” includes all forms of prions causing all or any of these diseases or others in any animals used--and in particular in humans and domesticated farm animals. The pathogen can be virtually any pathogen for which genetic information (e.g., gene sequences) is available.

In preferred embodiments, modified APCs are contacted to T cells in a culture medium for a period sufficient to, upon binding of T cells to APCs expressing relevant antigens, induce a morphological change in the modified APCs such that the contacted modified APCs take on a morphology of a more mature APC. As used herein, the term “mature APC” refers to the state of an APC following in vitro or in vivo differentiation in the presence of appropriate stimuli such that the mature APC has the capacity to initiate or engage in an immune response. Mature APCs (“mAPCs”) express CD40, CD54, CD80, CD83, CD86, CCR7, ICAM-1, CD1a, and high levels of MHC class II, as measured by monoclonal antibody (mAb) staining and flow cytometric analysis.

Induced maturation in the APCs can be detected using, for example, a colorimetric or fluorometric assay. For example, antibody-mediated detection can be performed using antibodies specific for a particular marker in combination with any fluorophore or other label suitable for the detection and sorting of cells (e.g., fluorescence-activated cell sorting (FACS)). Antibody/fluorophore combinations to specific markers include, but are not limited to, fluorescein isothiocyanate (FITC) conjugated monoclonal antibodies against HLA-G (available from Serotec, Raleigh, N.C.), CD10 (available from BD Immunocytometry Systems, San Jose, Calif.), CD44 (available from BD Biosciences Pharmingen, San Jose, Calif.), and CD105 (available from R&D Systems Inc., Minneapolis, Minn.); phycoerythrin (PE) conjugated monoclonal antibodies against CD44, CD200, CD117, and CD13 (BD Biosciences Pharmingen); phycoerythrin-Cy7 (PE Cy7) conjugated monoclonal antibodies against CD33 and CD10 (BD Biosciences Pharmingen); allophycocyanin (APC) conjugated streptavidin and monoclonal antibodies against CD38 (BD Biosciences Pharmingen); and Biotinylated CD90 (BD Biosciences Pharmingen). Other antibodies that can be used include, but are not limited to, CD133 APC (Miltenyi), KDR-Biotin (CD309, Abcam), CytokeratinK-Fitc (Sigma or Dako), HLA ABC-Fitc (BD), HLA DRDQDP-PE (BD), β-2-microglobulin-PE (BD), CD80-PE (BD) and CD86-APC (BD).

The T cell activation process requires two signals to occur at once. First, the T cell must encounter its cognate antigen after it has been properly processed, which includes proteolysis into the appropriate peptides, and displayed on the surface of an APC. Second, the APC must also display the matching HLA type for the T cell. Thus measuring which antigens activate a T cell cannot be done by binding T cells to a surface displaying antigens. If both of the above conditions are met, the T cell will be activated and it will, in turn, induce changes in the APC. APCs which have been induced during the activation of T cells (otherwise known as mature APCs or “mAPCs”) can be separated from “un-induced” (non-activated) APCs using any appropriate method. In preferred embodiments, mAPCs are separated APCs from those that remain unmodified using a technique such as flow cytometry, biochemical sorting, or fluorescence-activated cell sorting (FACS). FACS is a well-known method for separating particles, including cells, based on the fluorescent properties of the particles (Kamarch, 1987, Methods Enzymol., 151:150-165). Other separating or sorting techniques appropriate for use according to the methods described herein include, without limitation, immunopanning, affinity chromatography, and magnetic activated cell sorting (MACS), including antibody-mediated magnetic cell sorting, and other magnetic (immuno-magnetic) techniques. In a typical antibody-mediated magnetic cell sorting procedure, cells are contacted with a specific primary antibody, and then captured with a secondary anti-immunoglobulin reagent bound to a magnetic bead. The adherent cells are then recovered by collecting the beads in a magnetic field.

In some cases, a cytokine secretion assay is performed to separate APCs which have been induced during the activation of T cells (otherwise known as mature APCs or “mAPCs”) from “un-induced” (non-activated) APCs. For example, any appropriate method for detecting secretion of one or more cytokines can be performed. Cytokines that can be detected include, without limitation, IFNγ, GM-CSF, IFNα, IL-2, IL-4, IL-5, IL-10, IL-12, IL-13, and IL-17. Magnetic-based cell sorting techniques (e.g., MACS) or FACS analysis can be used in connection with such cytokine “capture assays” to detect and separate (e.g., isolate, sort, enrich) viable cytokine-secreting cells with single-cell sensitivity. Briefly, in cytokine secretion or “capture” assays, a cytokine secreted by the cell is captured either by a matrix in which the cell is embedded, by particle-attached heterobispecific antibodies, or by the use of magnetic nanoparticles bound to anti-mouse IgG for specific binding to mouse antibodies against the cytokine of interest. Other detection methodologies such immunoassays (e.g., ELISA) can be used to detect secretion of one or more cytokines.

In some cases, other phenotypic properties of activated and non-activated APC cell populations are assessed using methods such as microscopy, in situ hybridization, in situ polymerase chain reaction (PCR), standard flow cytometry methods, enzyme-linked immunosorbent assay (ELISA), and enzyme-linked ImmunoSpot (ELISPOT) assay. In some cases, it may be advantageous to assess cell viability and proliferation potential using standard techniques known in the art, such as trypan blue exclusion assay, fluorescein diacetate uptake assay, propidium iodide uptake assay (to assess viability); and thymidine uptake or MTT cell proliferation assays (to assess proliferation).

Any appropriate method of detecting activated APCs can be used with the steps described herein. For example, it can be advantageous to detect APC lysis or cytotoxicity resulting from T-cell targeting. Markers useful for detecting APC lysis and T-cell related cytotoxicity include, without limitation, antibodies or reagents that detect T-cell effector molecules, such as caspases, Granzymes (A, B, H), perforin, annexin, and apoptotic pathway factors. In some cases, APC activation is detected by capturing cytokines secreted by T-cells as a result of their activation on cell surface of or inside a modified APC. See Example 1 in the following Examples. Standard cell sorting techniques can be used.

Following separation of APCs induced during the activation of T cells from non-activated APCs, the collected pool of APCs that were enriched for successful T cell activation (i.e., mAPCs) are lysed and the nucleic acids are extracted. In some cases, nucleic acids are also isolated from the non-activated APC population. Any appropriate method can be used to detect nucleic acids. For example, polymerase chain reaction (PCR) and DNA sequencing techniques including but not limited to Ion torrent, MiSeq, and HiSeq can be used. In preferred embodiments, PCR amplification is used to amplify nucleic acids from the extracted DNA exogenous nucleic acid sequences that were initially introduced to the APCs.

Any appropriate method can be used to analyze the resulting amplified DNA. In preferred embodiments, one or more genomic sequencing methods such as “next generation DNA sequencing” are used for such analysis. The terms “DNA sequencing” and “sequencing” as used herein refer to methods by which the identity of at least 10 consecutive nucleotides (e.g., the identity of at t least 50, at least 100, at least 200, or at least 500 or more consecutive nucleotides) of a nucleotide sequence are obtained. As used herein, the term “next generation sequencing” refers to DNA sequencing methodologies that share the common feature of massively parallel, high-throughput strategies, with the goal of lower costs in comparison to older sequencing methods (see, e.g., Voelkerding et al., Clinical Chem., 55: 641-658, 2009; MacLean et al, Nature Rev. Microbiol, 7-287-296; each herein incorporated by reference in their entirety). Next generation sequencing (NGS) methods can be broadly divided into those that typically use template amplification and those that do not. Amplification-requiring methods include commercially available platforms such as pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos Biosciences, and emerging platforms commercialized by VisiGen, Oxford Nanopore Technologies Ltd., Life Technologies/Ion Torrent, and Pacific Biosciences, respectively.

Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer, and are described in Gudmundsson et al (Nat. Genet. 2009 41:1122-6), Out et al (Hum. Mutat. 2009 30:1703-12) and Turner (Nat. Methods 2009 6:315-6), U.S. Patent Application Pub nos. US20080160580 and US20080286795, U.S. Pat. Nos. 6,306,597, 7,115,400, and 7232656. 454 Life Science platforms include, e.g., the GS Flex and GS Junior, and are described in U.S. Pat. No. 7,323,305. Platforms from Helicos Biosciences include the True Single Molecule Sequencing platform. Platforms for ion semiconductor sequencing include, e.g., the Ion Torrent Personal Genome Machine (PGM) and are described in U.S. Pat. No. 7,948,015. Platforms for pryosequencing include the GS Flex 454 system and are described in U.S. Pat. Nos. 7,211,390; 7,244,559; 7264929. Platforms and methods for sequencing by ligation include, e.g., the SOLiD sequencing platform and are described in U.S. Pat. No. 5,750,341. Platforms for single-molecule sequencing include the SMRT system from Pacific Bioscience and the Helicos True Single Molecule Sequencing platform.

While the automated Sanger method is considered as a ‘first generation’ technology, Sanger sequencing including the automated Sanger sequencing, can also be employed according to the methods provided herein. Additional sequencing methods that comprise the use of developing nucleic acid imaging technologies (e.g., atomic force microscopy (AFM) or transmission electron microscopy (TEM)) are also encompassed by the methods provided herein.

In other cases, APC activation is detected by performing a T-cell mediated cell death assay. See Example 2 in the following Examples. In this manner, this disclosure provides another method for detecting antigen-targeting of target cells by T-cells. For example, an assay that detects uptake of a cell-impermeable fluorescent dye such as propidium iodide (PI) or ethidium homodimer can be performed to selectively detect T-cell mediated cell-death. Such fluorescent dyes are a small fluorescent molecules that selectively stains cells with compromised membrane integrity but cannot passively traverse into cells having an intact plasma membrane. PI uptake versus exclusion can be used to discriminate dead cells, in which plasma membranes become permeable, from live cells with intact membranes. Fluorescence from DNA binding dyes such as PI upon uptake into dead cells can be detected using a flow cytometry protocol or any other high throughput screening method. In some cases, the PI uptake assay can be performed in conjunction with staining of surface antigens with antibodies. In some cases, T-cell mediated cell death assay is detected by measuring lactate dehydrogenase (LDH), a stable cytoplasmic enzyme which is present in all cells but only released when the plasma membrane is damaged. Colorimetric assays can be performed to measure LDH levels, which are proportional to the number of dead or damaged cells in a sample.

In some embodiments, the method further includes identifying genes enriched in mAPCs. For example, analysis can be performed to identify genes enriched in mAPCs relative to non-activated APCs, thus revealing proteins that triggered T cell activation.

In some embodiments, the method further includes determining frequency of antigen-specific T cells in uncontacted T cell population.

In some embodiments, the method further includes identifying T cell and/or T cell receptor responsible for antigen targeting.

In another aspect, provided herein is a method for detecting epitope-specific T cells and target epitopes of reactive T cells. As used herein, the term “epitope” refers to a region of a polypeptide that exhibits antigenic features and serves for example as a recognition site of T cells or immunoglobulins. Antigens and epitopes can also encode wild-type or variant (e.g., mutated) nucleotides and amino acids expressed in normal tissue and/or diseased tissue (e.g., tumor). In terms of the methods provided herein, epitopes include such regions of polypeptides which are recognized by immune cells such as, for example, CD4⁺ T helper cells, CD8⁺ cytotoxic T cells, CD161⁺ NKT cells, or CD4⁺CD25⁺ regulatory T-cells. An epitope can comprise 3 or more amino acids. Usually an epitope consists of at least 5 to 7 amino acids or, more often, of at least 8-11 amino acids, or of more than 11 amino acids, or of more than 20 amino acids, less frequently even of more than 30 amino acids. The term “epitope” comprises both linear and a steric conformation being unique for the epitope. The steric conformation results from the sequence of the amino acids in the region of the epitope.

In another aspect, provided herein is a method for obtaining a cellular immune profile of an individual.

The methods described herein can be carried out using a computer programmed to receive data (e.g., data from a subject's cellular immune response profile) and capable of displaying the information via a user interface.

After information regarding a subject's cellular immune response profile is reported, a professional can take one or more actions that can affect patient care. For example, a medical professional can record the information in a subject's medical record and/or in an electronic database. In some cases, a medical professional can record that the subject may or may not have an infection or illness associated with one or more specific antigens, or otherwise transform the patient's medical record, to reflect the patient's medical condition. In some cases, a medical professional can review and evaluate a patient's medical record, and can assess multiple treatment strategies for clinical intervention of a patient's condition.

A professional (e.g., medical professional) can communicate information regarding cellular immune response analysis to a subject or a subject's family. In some cases, a professional can provide a subject and/or a subject's family with information regarding a therapy, including treatment options and potential side effects, that may be effective given a subject's particular epitope profile. In some cases, a professional can provide a copy of a subject's medical records to communicate information regarding cellular immune response analysis (e.g., epitope profile) and/or disease states to a specialist.

A professional (e.g., research professional) can apply information regarding a subject's epitope profile to advance research into cellular immune responses. For example, a researcher can compile data on the presence of a particular epitope profile with information regarding the efficacy of a particular therapy or side effects associated with a particular therapy. In some cases, a research professional can obtain a subject's epitope profile information to evaluate the subject's enrollment, or continued participation in a research study or clinical trial. In some cases, a research professional can communicate a subject's epitope profile information to a medical professional, or can refer a subject to a medical professional for clinical assessment and/or treatment.

Any appropriate method can be used to communicate information to another person (e.g., a professional), and information can be communicated directly or indirectly. For example, a laboratory technician can input epitope profile information into a computer-based record. In some cases, information can be communicated by making a physical alteration to medical or research records. For example, a medical professional can make a permanent notation or flag a medical record for communicating information to other medical professionals reviewing the record. Any type of communication can be used (e.g., mail, e-mail, telephone, and face-to-face interactions). Information also can be communicated to a professional by making that information electronically available to the professional. For example, information can be placed on a computer database such that a medical professional can access the information. In addition, information can be communicated to a hospital, clinic, or research facility serving as an agent for the professional.

### Articles of Manufacture

This document also provides articles of manufacture that can include, for example, materials and reagents that can be used to determine whether a subject has a cellular immune response to certain antigens. An article of manufacture can include, for example, a control population of T cells. The article of manufacture can also include instructions for use in practicing a method for classifying antigens as recognized by a subject's T cell population as provided herein. An article of manufacture may further comprise one or more nucleic acids and instructions for modifying antigen presenting cells as described herein. Optionally, reagents such as transfection reagents or detection reagents may be provided separately from the other kit components.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g., via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; and Berg et al. (2002) Biochemistry, 5^{th} Ed., W. H. Freeman Pub., New York, N.Y., all of which are herein incorporated in their entirety by reference for all purposes.

“Determining,” “measuring,” “assessing,” “assaying” and like terms are used interchangeably and can include both quantitative and qualitative determinations. Assessing may be relative or absolute. “Assessing the presence of” includes determining the amount of something present, as well as determining whether it is present or absent.

Note that as used herein and in the appended claims, the singular forms “a,” “an,” and “the” include plural referents unless the context clearly dictates otherwise. Thus, for example, reference to “an array” refers to one or more such arrays, and reference to “the method” includes reference to equivalent steps and methods known to those skilled in the art, and so forth.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Although the embodiments are described in considerable detail with reference to certain methods and materials, one skilled in the art will appreciate that the disclosure herein can be practiced by other than the described embodiments, which have been presented for purposes of illustration and not of limitation. Therefore, the scope of the appended claims should not be limited to the description of the embodiments contained herein.

### EXAMPLES

### Example 1—Target APC Detection by Capturing Cytokines on APC Cell Surface

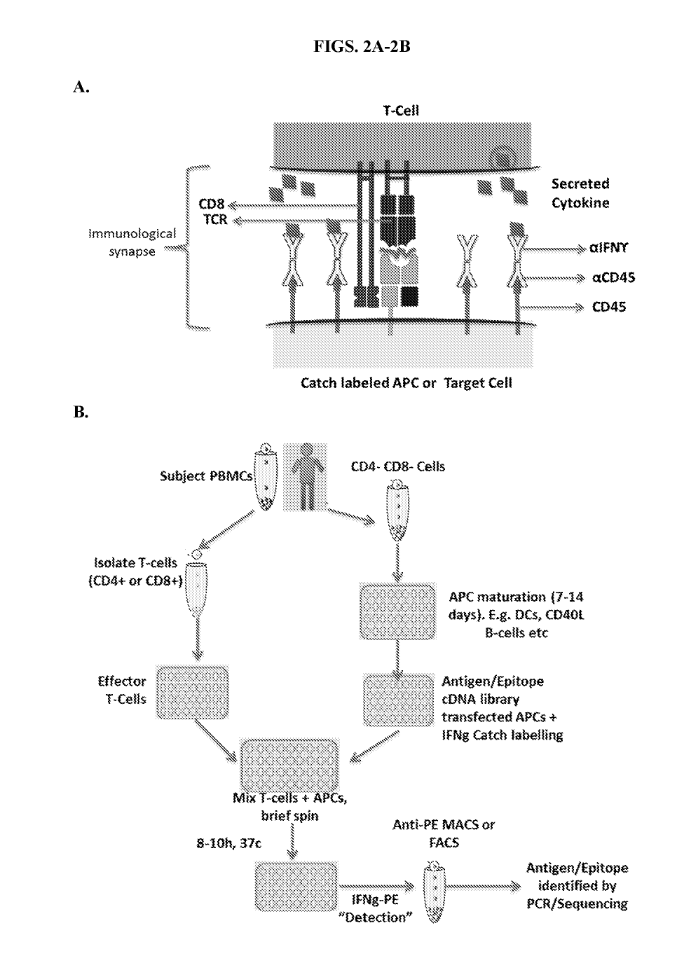

As shown in FIG. 2, antigen-transfected APCs are specifically pre-labeled using a bispecific cytokine capture reagent such as IFNγ. As shown in the example workflow, APCs isolated from a donor are matured in culture for 7-10 days, and are then mixed with purified T-cells (CD8 or CD4) from the same donor (i.e., autologous T cells), or using cells from an artificial genetically engineered antigen presenting cell line. T-cells activated by the antigen will target the APCs secreting cytokines such as IFNγ, which are captured on the APC cell surface via capture reagents such as IFNγ. Cytokine-captured APCs are sorted and isolated using magnetic or fluorescence-based sorting techniques in order to isolate nucleic acids corresponding to the antigen. Such nucleic acids are amplified and identified using next-generation sequencing.

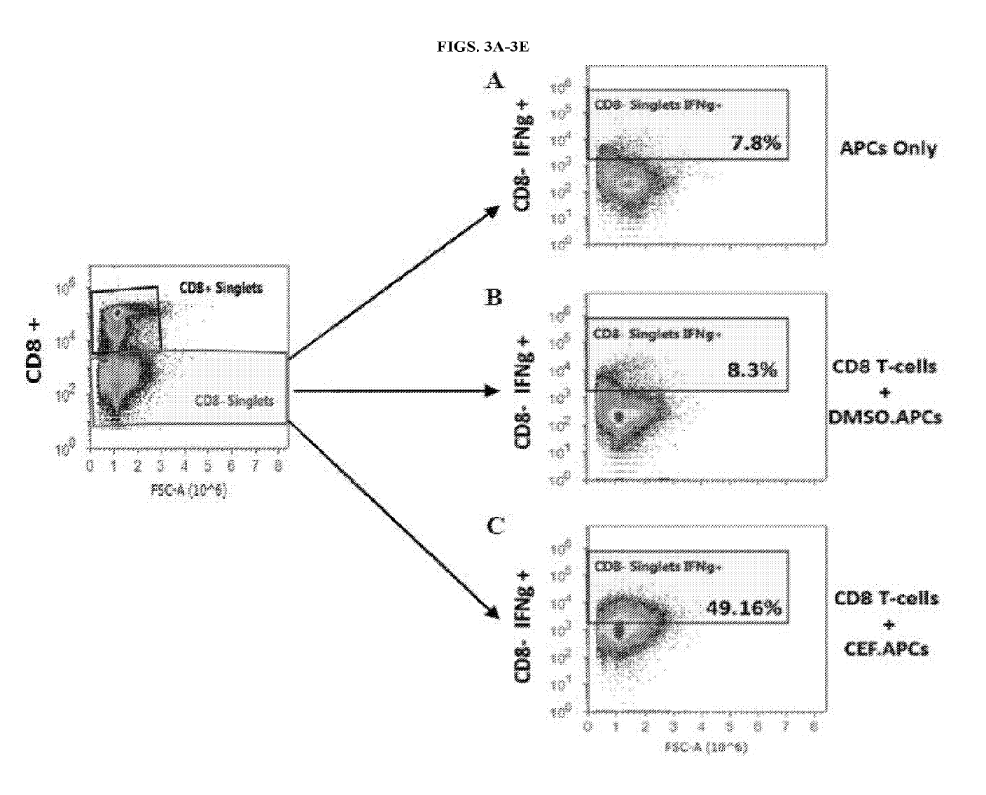

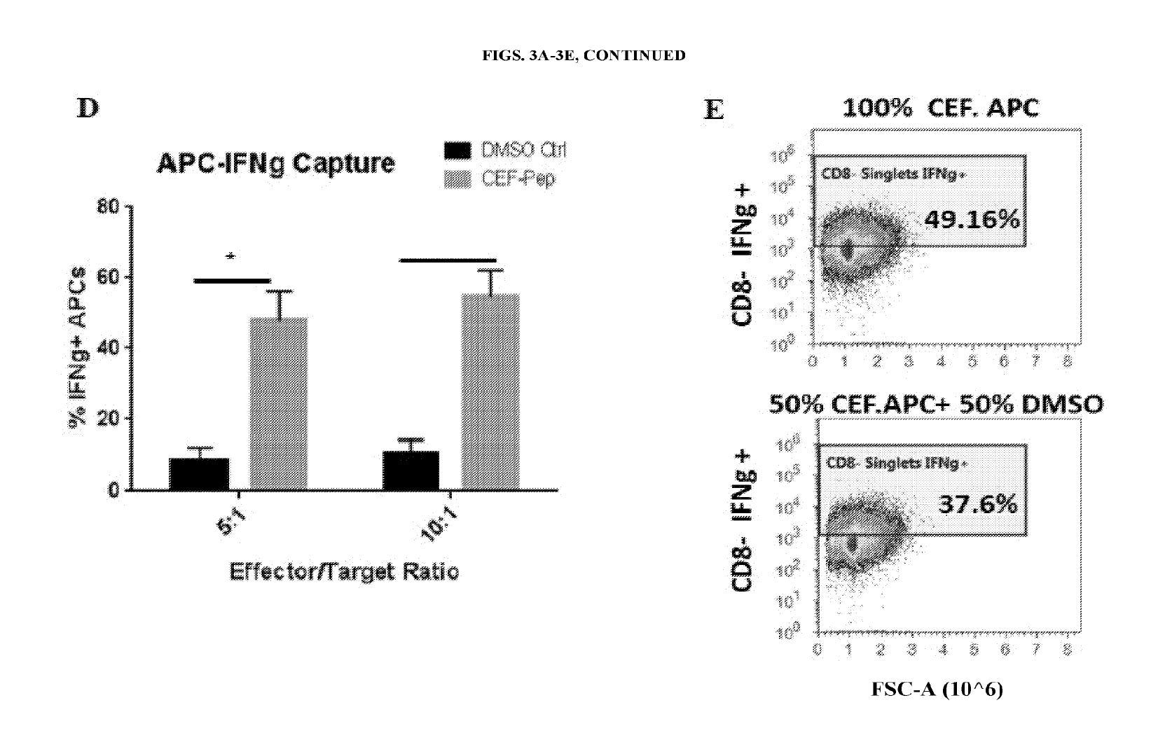

Exemplary data for the “captured cytokines” approach to target APC detection is presented in FIGS. 3A-3E. Donor APCs were matured, labeled with commercial IFNγ catch antibody (Miltenyi Biotec) and mixed with autologous CD8+ T-cells for 8 hours at 37° C. Cells were then washed and labeled with anti-CD8 antibody and anti-IFNγ antibody. As shown in FIGS. 3A-3C, APCs with cytokine captured by FACS were six-fold higher when pulsed with the CEF (CMV+EBV+Flu) viral peptide pool (C) as compared to APCs alone (A) or APCs pulsed with DMSO control (B). As shown in FIG. 3D, background cytokine captured did not increase as a function of T-cell number. FIG. 3E demonstrates cytokine capture on APC-cell surfaces when (i) CD8+ T-cells target 100% of APCs pulsed with CEF-peptide pool (Top panel), and (ii) when only 50% of APCs were CEF-pulsed, but the remainder of the cells were DMSO “cold” targets (Bottom panel). These data demonstrate that the number of T-cells and number of target-APCs are dose dependent. When the number of true-target APCs is reduced by half by artificially making a mix population of targets+non-targets (50% each), the signal drops (to 37%) as opposed to when all the APCs are pulsed with peptides (100% targets=50%). Thus, the assay may be single-cell specific (we are still doing experiments to confirm the limit of detection) and the carryover of captured cytokine onto other cells is minimal. This will be important when detecting antigens using sequencing. Cytokine capture on APC-cell surface was not saturating, and was dependent on number of true target cells. These results demonstrate that the assay is single-cell specific and there is minimal carryover of captured cytokine onto other cells.

### Example 2—Target Antigen-APC Detection by T-Cell Mediated Cell Death Assay

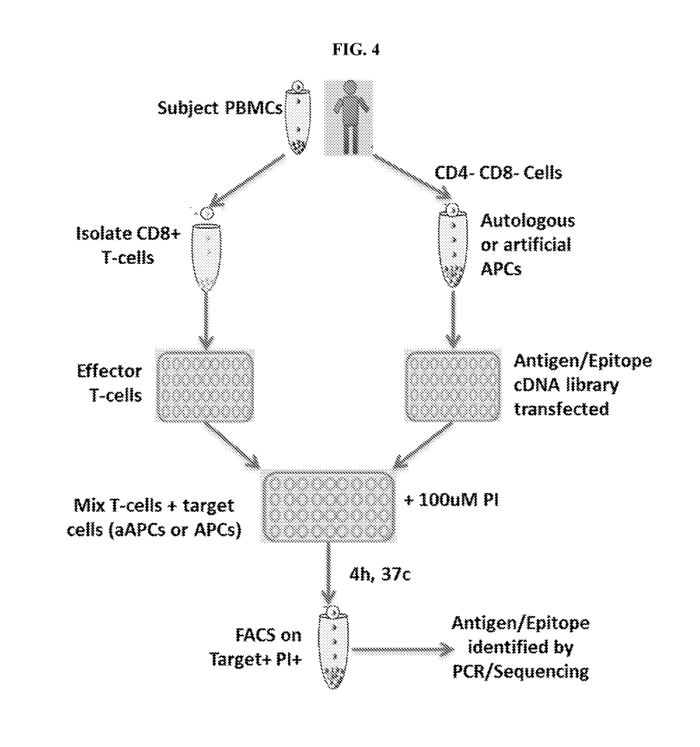

FIG. 4 shows an exemplary workflow for PI-death assay on antigen transfected APC cell surface. APCs isolated from a donor, or an HLA-matched artificial APC (aAPC) cell line such as K562, are matured in culture for 7-10 days. The matured cells are then mixed with purified CD8 T-cells. CD8 T-cells activated by the antigen will target the APCs and initiate target cell death. Membrane permeable DNA-intercalating fluorescent dyes such as PI at high concentration enter the compromised cell membranes, and cells labeled with such dyes are sorted or separated using flow cytometry cell sorting (FACS). FACS-sorted antigen-APCs are used to isolate nucleic acid corresponding to the antigen and identified by PCR/next-generation sequencing.

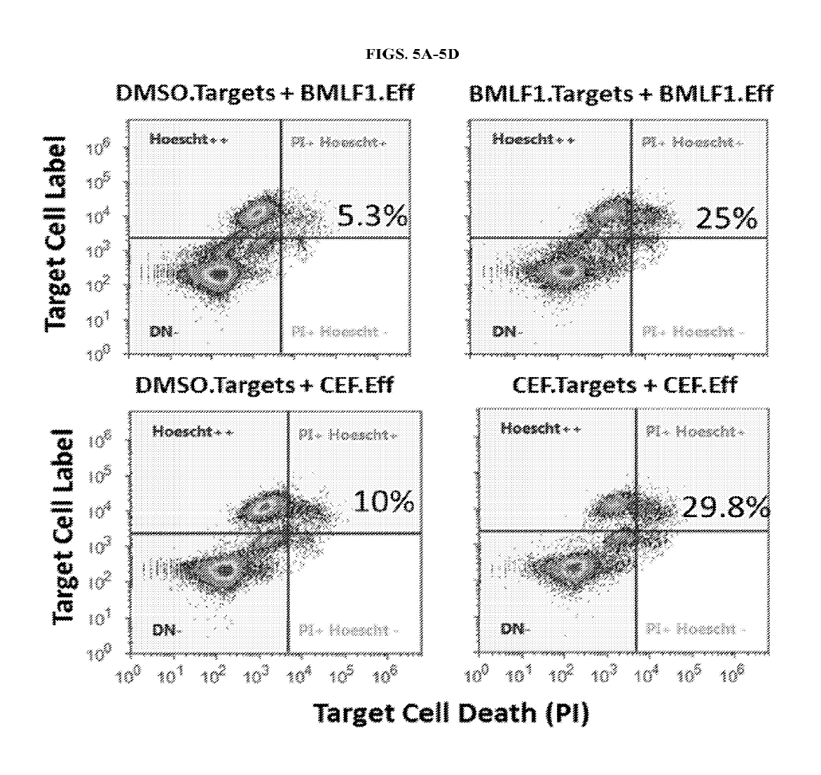

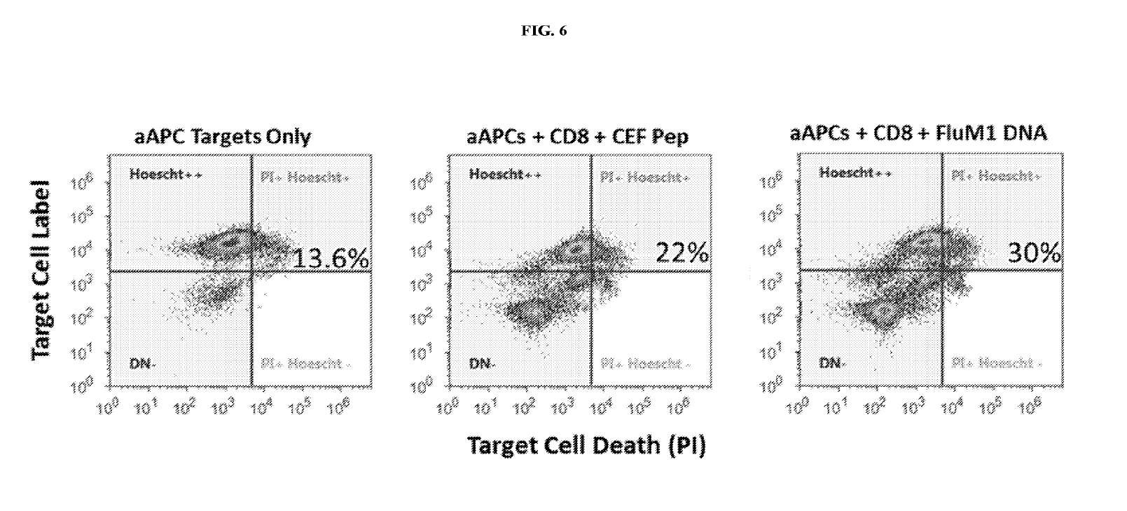

FIG. 5 shows representative data for a propidium iodide (PI)-death assay for antigen-transfected APC cell surface. In this figure, T-cell mediated target cell death is indicated by the PI+Hoescht+quadrant.

We claim:

1. A method of classifying antigens recognized by a T cell population, the method comprising
(a) contacting antigen presenting cells (APCs) modified to express at least one preselected target antigen to a plurality of T cells, wherein contacting occurs under conditions sufficient to allow binding between a T cell that specifically recognizes the at least one preselected target antigen expressed by the modified APCs;
(b) separating target antigen-expressing APCs activated by the contacting;
(c) amplifying nucleic acids isolated from the activated APC cells; and
(d) detecting nucleotide sequences encoding preselected target antigens from the amplified nucleic acids, wherein a preselected target antigen is classified as being recognized by T cells of the plurality if a nucleotide sequence encoding the preselected target antigen is detected among the amplified nucleic acids.

2. The method of claim 1, wherein binding between the modified APCs and the T cell comprises formation of an immune complex between the modified APCs and a T cell receptor on the T cell that recognizes the at least one preselected target antigen.

3. The method of claim 1, wherein target antigen-expressing APCs activated by the contacting are separated from non-activated APCs and maturing APCs.

4. The method of claim 1, further comprising measuring a level of a cell surface antigen on contacted APCs relative to uncontacted APCs.

5. The method of claim 1, wherein separating comprises fluorescence-based sorting or magnetic sorting.

6. The method of claim 1, wherein separating comprises detecting contacted APC secreting one or more cytokines.

7. The method of claim 6, wherein the secreted cytokine is selected from the group consisting of IFNg, GM-CSF, IFNa, IL-2, IL-4, IL-5, IL-10, IL-12, IL-13, and IL-17.

8. The method of claim 1, wherein the modified APCs present the at least one preselected target antigen.

9. The method of claim 8, wherein the modified APCs are obtained by recombinant techniques.

10. The method of claim 1, wherein the antigen presenting cell is selected from the group consisting of a dendritic cell, a macrophage, a monocyte, and a B cell.

11. The method of claim 1, wherein the at least one preselected target antigen is derived from at least one of a tumor cell, a virus, a bacterium, a fungus, a yeast, and a parasite.

12. The method of claim 1, wherein the at least one preselected target antigen is a pathogen associated target antigen.

13. The method of claim 12, wherein the pathogen is a virus, bacterium, fungus, yeast, or parasite.

14. The method of claim 13, wherein the virus is selected from the group consisting of cytomegalovirus (CMV), adenovirus, Epstein Barr virus (EBV), respiratory syncytial virus (RSV), herpes simplex virus 6 (HSV6), parainfluenza 3, influenza B, BK virus, and JC virus.

15. The method of claim 1, wherein the at least one preselected target antigen is a tumor associated antigen or an auto-immune antigen.

16. The method of claim 1, wherein the at least one preselected target antigen comprises an epitope.

17. The method of claim 1, wherein the T cell population is obtained from a human individual.

18. The method of claim 1, wherein the T cell population is produced in vitro.

19. The method of claim 1, wherein detecting nucleotide sequences comprises DNA sequencing.

20. The method of claim 1, further comprising determining the relative abundance of antigen-specific T cells in the uncontacted T cell population.

21. The method of claim 1, wherein the antigen presenting cells (APCs) are derived from a healthy donor.
